(19) United States
(12) Patent Application Publication
(10) Pub. No.: US 2003/0028154 A1
(43) Pub. Date: Feb. 6, 2003

(54) Polymer hypodermic needle and process for producing same design and process for making all-plastic molded-in-one piece hypodermic needle

(76) Inventor: Milton Ross, Wayne, PA (US)

Correspondence Address: DANIEL E KRAMER, 2009 WOODLAND DRIVE, YARDLEY, PA, 19067, US

(21) Appl. No.: 10/208,572

(22) Filed: Jul. 30, 2002

Related U.S. Application Data
(60) Provisional application No. 60/309,082, filed on Jul. 31, 2001.

(51) Int. Cl. A61M 5/32; B29C 45/00

(52) U.S. Cl. 604/272; 606/185; 249/115; 249/134; 264/328.1

(57) ABSTRACT

A polymer mixture, a mold design including a porous steel part and a process for producing a one piece polymer hypodermic tip having a stiff needle part of uniform small diameter and a length exceeding 0.4 inches.

### I. PRIORITY CLAIM

Applicant claims priority based on Provisional Patent Application filed Jul. 31, 2001 and bearing Serial No. 60/309,082.

### II. BACKGROUND

1. Field of the Invention

The invention is directed to a molded-in-one-piece hypodermic needle having an integral polymer hub, barrel and at least one thin walled part or needle having a uniform outside diameter. Such a complete assembly is frequently referred to in the industry as a ‘tip.’ Colloquially, however, both the assembly and the thin needle-like part are referred to as a ‘needle.’

2. Prior Art

Hypodermic needles are thin tube devices, generally referred to in the trade as “tips” that are intended to be used with and fastened to liquid dispensing devices. In one very common version, the tips include a barrel having at one end an interrupted thread connector called a Luer. The Luer is designed to make connection with the dispensing device. A needle of stainless steel or a polymer such as polycarbonate or polypropylene is cemented into the end of the barrel opposite from the Luer. Examples of liquid dispensing devices are mechanical dispensers used for placing cements or fluxes on a printed circuit boards and syringes for injecting measured quantities of medicines into humans. The tips are made in a wide range of needle outside diameters (OD) and range in length from 0.25 inches to 1.5 inches. The diameters are stated in wire gage sizes. One manufacturer provides the following brief table of needle inside and outside diameters:

| Gage | OD(Inches) | ID(Inches) | Wall |
|---|---|---|---|
| 14 | 0.072 | 0.060 | 0.006 |
| 15 | 0.065 | 0.054 | 0.0055 |
| 18 | 0.050 | 0.033 | 0.0085 |
| 20 | 0.036 | 0.023 | 0.0065 |
| 21 | 0.032 | 0.020 | 0.006 |
| 22 | 0.028 | 0.016 | 0.006 |
| 23 | 0.025 | 0.013 | 0.006 |
| 25 | 0.020 | 0.010 | 0.005 |
| 27 | 0.016 | 0.008 | 0.004 |
| 30 | 0.012 | 0.006 | 0.003 |
| 32 | 0.009 | 0.004 | 0.0025 |

IV. In the past, unsuccessful efforts have been made to mold a uniformly thin needle, barrel and Luer assembly in one piece. Manufacturers have made an effort to overcome these failures to produce an integral needle of uniformly small diameter by offering a so-called “tapered tip” that tapers uniformly from the barrel to the tip. (EFD Company catalog; 977 Waterman Ave, East Providence, R.I. 02914-1378) However, this does not meet the needs of many manufacturing operations where the needle part must have a uniform small diameter.

### V. ADVANTAGES OF ONE PIECE MOLDING

A fully formed single piece tip has obvious advantages in production speed and simplicity thereby providing the advantages of lower cost. For example, both stainless steel and plastic needles that are to be glued into the barrel need to be cut and finished to remove burrs, beveled or chamfered, polished and the steel passivated. By contrast, one piece molding automatically gives a highly polished beveled or chamfered or straight across cut and no further passivation is required.

VI. There are many additional benefits arising from molding a uniformly narrow needle tip in one piece:

First is consistency of the product;

Second is lower resistance to flow of an all-in-one needle compared to an assembled needle. This allows lower feed pressures and faster deposition of the required material on the circuit board or fabrication thereby improving the overall efficiency of the device or circuit board production. The lower flow resistance arises from a series of factors.

A) The transition from the larger internal diameter of the shank to the much smaller internal diameter of the needle is smooth and streamlined compared with the abrupt entrance to the needle in the assembled product.

B) The one piece construction allows a significantly shorter small diameter flow path (compare FIGS. 2A and 2B).

C) Plastic needle interiors have smoother inner surfaces and higher lubricity and offer lower resistance to liquid flow than the rough surfaces inherent in the interior of steel needles.

D) Plastics can have lubricating additives blended into the composition prior to molding that further reduce the resistance to flow of fluids drawn into or dispensed by the needle.

Third, the dispensing equipment is easier to control because of the lower feed pressures required, thereby improving the consistency and size of the drops dispensed.

Fourth, the smooth flow transition in the one piece needle flow pattern that avoids trapping adhesives. This reduces or eliminates dead zones within the barrel and needle that causes premature curing and activation of catalysts that cause clogging and reduced flow, thereby requiring early needle replacement.

Fifth, the cost of manufacturing can be sharply reduced.

A) The manufacturing process can be simplified, whereby the steps of sealing the needle into the barrel by cementing, gluing, heat welding or press-fitting a separate plastic or metal needle into the barrel are eliminated.

B) The cost of the cement is eliminated.

C) The cost of the machinery to automate the needle insertion and gluing process that has to be amortized in the cost of each needle is eliminated.

D) The cost of the thin walled separately produced tubing is eliminated. Addressing only the 0.25 inch extra length of thin walled tube required in a million needle run can become significant, this extra buried length amounts to almost 21,000 extra feet that must be purchased.

All of these, when factored into the final cost of the product, provide a more competitive product.

VII. Although the main application of the plastic needles is expected to be industrial, there are medical applications for use in the laboratory distinct from those applications requiring penetrating of human skin for injecting fluids into the body or withdrawing fluids from the body.

VIII. Such advantages of the integral molded tip are important in industries such as the semiconductor industries where printed circuit (PC) board assemblies require securing components or masks to the PC boards. One such function requires deposition of uniform patterns of solder paste or adhesive application to the PC board. The precision dots of such materials must be precisely located to assure uniformity in the final product. This requires needles that maintain their shape. Further, weight repeatability, dot shape and tight size tolerances are required to reduce problems arising from slumping, stringing and tailing. These are best achieved by ensuring precise and repeatable ‘break-away’ of the needle from the deposited dot of deposited material. The higher lubricity of polymers over that of steel augments this break away characteristic. Unique flux or cement patterns are sometimes required. These are readily secured with special needle shapes that can be easily secured with mold modification such as by varying the mandril shape. That determines the interior needle bore shape.

IX.

Integral molded tips offer all these advantages over tips having glued-in-place steel or polymer needles.

### X. SUMMARY OF THE INVENTION

Integrally molded hypodermic tips having a needle portion of uniform diameter equal to or less than 0.072 inches and processes including polymer composition and mold design for making such tips.

### XI. OBJECTS AND ADVANTAGES

a. It is an object of the invention to provide a unique mold for producing a one piece thermoplastic polymer tip having a needle portion of uniformly small diameter.

b. It is a further object to provide such a mold where the mold portion within which the needle part is cast is formed of a different material from the portion within which the barrel and Luer are cast.

c. It is a further objective to provide such a mold where at least the needle portion of the mold is formed of porous steel.

d. It is a further objective to provide such a tip formed of a polymer having fine particles embedded therein to stiffen and improve the physical properties of the needle part whereby improved needle part straightness, stiffness, rigidity and concentricity is secured on removal of the tip from the mold.

e. It is a further objective to provide such improved stiffness employing a polymer having embedded therein particles of the class “Nano-Clay” provided by NanoCor Industries, Honeywell, Eastman or Ticona, thereby forming a matrix of the particles within the polymer.

f. It is a further objective that the melting point of the particles within the matrix be much higher than the melting point of the polymer.

g. It is a further objective to provide such a needle employing a polymer having improved flow characteristics for fluid passing through the needle.

h. It is a further objective to provide such a needle where the flow characteristics of the casting polymer are improved by addition of either or both Ticona “Ceramer TM” a Polyphenylene Sulphone (PPSO₂) and DuPont Fluoroguard “TM.

i. It is a further object to provide a barrel with two or more integrally molded needle parts.

j. It is a further object to provide tips where the dispensing end of the needle part has been formed into a specialized shape.

k. It is a further object to provide such a needle part where the needle part end applies material in the form of a ‘x’.

l. In a tip having multi-needle parts it is a further object to provide a turbulator positioned in the barrel between the Luer and the entrances to the needle parts to improve distribution among the multiple needle parts.

m. It is a further object to provide a mold for such a needle part that is split along the length of the needle.

n. It is a further object to provide a mold for such a needle part that causes the needle part to be formed with internal ribs or channels or external ribs or channels.

o. It is a further objective to provide a mold for such a needles with external ribs or channels.

p. It is a further object to provide such a tip having a needle part formed with polygonal internal and external crossections.

### XII. BRIEF DESCRIPTION OF THE DRAWINGS

FIG. 1A shows an outline in front elevation view of a molded one piece ‘tip’ or needle produced by the method and mold of the invention.

FIG. 1B shows the article of FIG. 1A in crossection.

FIG. 2A shows a Prior Are construction of a tip having a separate needle cemented into a polymer barrel.

FIG. 2B shows the much shorter restricted interior needle length and more streamlined flow pattern at the needle entrance secured with a one piece tip construction.

FIG. 3A is a crossectional illustration of the compound mold construction having an external lower or needle portion formed of one material and having an internal mandril concentrically positioned within the needle mold portion and an upper mold portion for the barrel and Luer or connection formed of a different material.

FIG. 3B shows the mold base plate having fastened therein one mandril that determines the interior shape and diameter of the needle part and with a tapered tip for mating with the tapered cavity in the upper body mandril.

FIG. 3C is an expanded view of the juncture between the lower mold portion and the upper mold portions showing the tapered end of the thin needle mandril secured in a recess in the end of the mandril for the body cavity.

FIG. 3D illustrates one form of an enlarged needle mandril end for securing specialized shapes.

FIG. 3E illustrates one form of a specialized shape of deposited material that can be secured by modifying either or both of the needle portion mold or the needle portion mandril.

FIG. 4A is a side elevation view of an all-in-one tip assembly or tip formed in one piece but having four integral needle parts of uniform small diameter positioned with their axes parallel to each other.

FIG. 4B is a crossection of the unit of FIG. 4A showing the internal flow passages and turbulating baffles provided to improve liquid flow distribution to the multiple needles.

FIG. 4C is an end view of the structures of FIGS. 4A and 4B showing the relative needle positions in the barrel as corners of a square.

FIG. 5A is a front elevation of a tip having multiple needle parts with the axes of the needle parts positioned parallel to each other and placed in a straight line orientation.

FIG. 5B is a crossectional elevation illustrating the interior flow pattern of the tip of 5A.

FIG. 5C shows a needle-end view of the tip of 5A showing the in-line needle positions in the barrel.

FIG. 6A shows a horizontally positioned mold where the parting line is along the centerline of the needle and barrel assembly.

FIG. 6B illustrates an enlarged section in the area where the needle and barrel mandrils are joined and integral instead of separate as in FIGS. 3A and 3C.

FIG. 6C shows a mold crossection at the needle area illustrating a mold construction that provides stiffening grooves and ribs in the needle bore and exterior.

FIG. 6D is a crossection of a needle having both internal and external ribs and channels.

FIG. 6E is a partial crossection of a needle formed with polygonal interiors and exteriors.

### DETAILED DESCRIPTION OF THE PREFERRED EMBODIMENT

XIII.

A major object of the invention is to produce an all-in-one hypodermic tip with a straight needle part having a substantially uniform diameter in the range from 0.072 to about 0.012. These can be used for purposes such as depositing fluxes or adhesives on printed circuit boards or penetrating rubber diaphragm or membrane type stoppers for test tube type vessels used for holding blood or solvents or medicines. Once having penetrated the membranes they are employed for withdrawing a measured amount from these vessels. The needle parts used for penetrating the membranes typically are in the larger range of outside diameters. The needles used for depositing the fluxes or cements are typically in the smaller range of outside diameters. To function correctly in these applications the needle parts must be straight and rigid. Polymers that can be employed in the molding process of the invention include polypropylene, Nylon, polyethylene and is 2-naphthalene Carboxylic Acid, 1(acetyloxy) polymer with 4-(acetyloxy) Benzoic Acid. This material has the trade name, Vectra A725 VD3049 and is obtainable from, among others Ticona Corp. (Formerly Hoechst Celanese Corp) at 90 Morris Ave; Summit, N.J. 07901.

XIV.

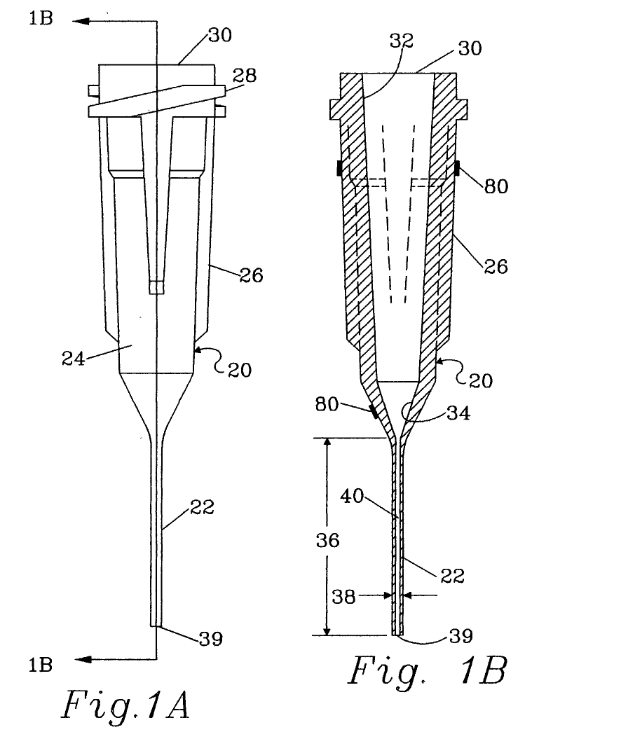

In FIG. 1A there is shown a front elevation of a hypodermic tip 20 having a barrel 24 and a large open end 30, generally employed as an inlet. Though this larger end 30 is used as an outlet where the tip is employed to withdraw fluid from a membrane sealed or other vessel, the end 30 will generally be referred to as an inlet with the understanding that it may be employed for either purpose. On the exterior of the end 30 is seen the interrupted threads of a Luer connector which is a substantially standard connector to hypodermic syringes. Threaded connections are also employed.

XV.

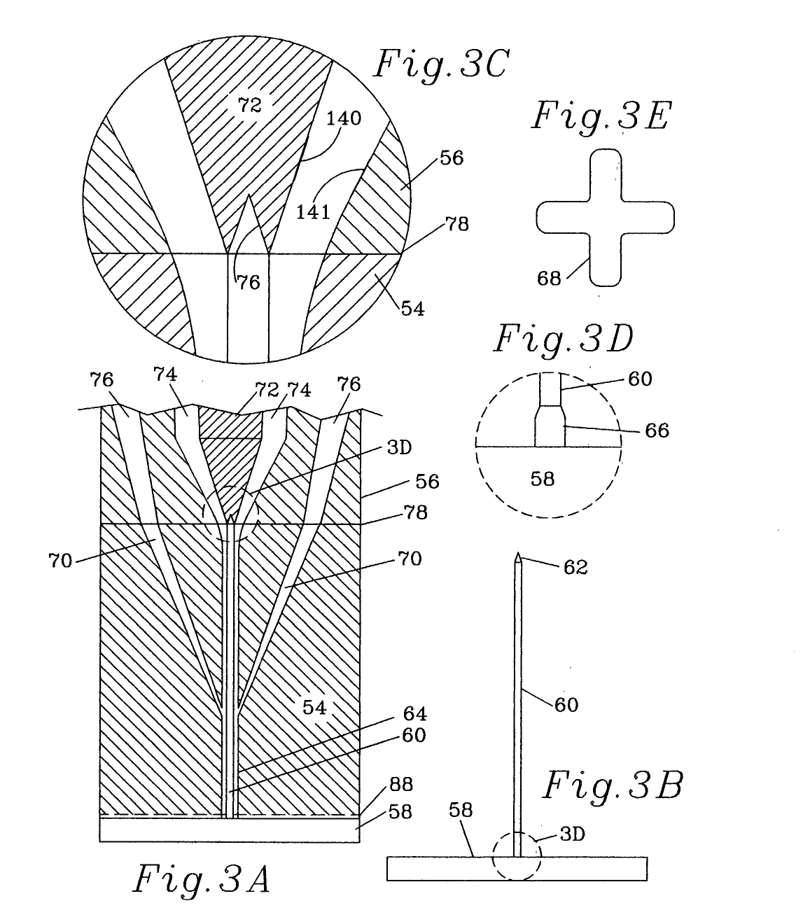

On the outside surface of barrel 24 four ribs 26 are provided to facilitate manually inserting and turning the tip to secure a connection to a dispensing or withdrawing device with the Luer 28 or with standard threads. At the end of barrel 24 opposite inlet connection end 30 there is an integrally molded needle part 22 having an end 39. The needle part 22 has a substantially uniform external diameter 38 and an inside bore 40 (FIG. 1B). The diameter of the bore 40 is substantially the same as the outside diameter of the mandril 60 (FIG. 3B).

XVI.

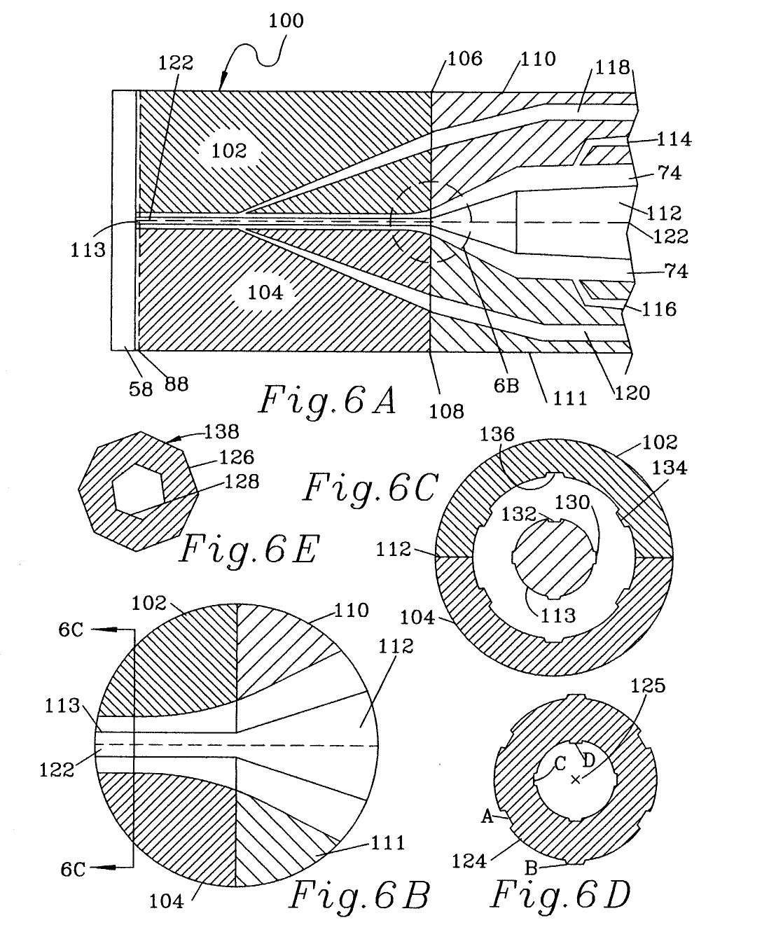

FIG. 1B illustrates a typical crossection of tip 20. The is shown the barrel end 30 and the barrel interior surface 32 as well as a typical location of gates 80 through which the molding material or mixture or matrix is injected into the mold that forms the tip 20 (see also FIGS. 3 and 6). In FIG. 1B the gates 80 are located on the ribs 26 of barrel 30 and on the transition between the barrel 30 and the needle part 22. The number of gates required depends on the size of the article being cast, the fluidity of the molding material, the difference between the mold temperature and the melting point of the casting material, the amount of mixture to be placed in the mold (mold volume per gate), the amount of residual gas in the mold to be displaced and the efficacy of the venting provisions and the distance from the gate to the most distant part of the mold to be filled. Since gates leave a small mark or bump on the cast product, they are usually placed where they do not affect the intended performance of the product. The location of gates is therefore subject to some experimentation. The number of gates required can be reduced by evacuating the mold prior to injection and by preheating the mold. Where needle 22 is required to be longer than approximately 0.5 inches, one or more gates may have to be placed along the length 36 of the needle part 22 to ensure a fully formed and filled needle part.

XVII.

In FIG. 1B the needle part 22 has length 36 and end 39. The interior 34 of the transition between barrel 30 and needle part 22 is highly streamlined to facilitate fluid flow of the material intended to be transmitted through the needle part 22.

XVIII.

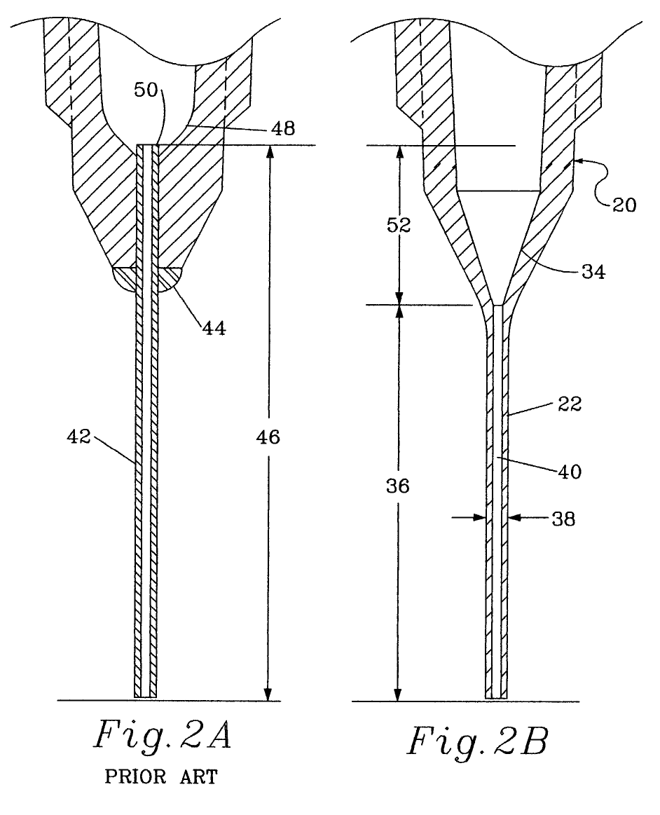

FIG. 2A compares Prior Art construction of a tip where the needle part 42 is prepared separately from the barrel and is cemented in place. There is shown a needle-less barrel part having interior 48. The separately manufactured needle part 48 is cemented into the barrel part with cement 44. The needle part 42 may be of material totally different from the barrel part. For example the needle 48 may be of drawn stainless steel or brass or Titanium tubing or of some thermoplastic polymer such as polycarbonate or a thermoset material having the desired chemical and physical characteristics. Polymer needles fabricated separate from the tip molding process may have had superior physical properties to prior art integrally cast needle parts. This is because the separately formed needle parts can be formed by extrusion and modified by drawing through dies or by other means prior to use to improve their physical properties, steps that are unavailable to the integrally cast needle part.

XIX.

The instant invention teaches the use of a base thermoplastic polymer such as polyethylene or polypropylene or Nylon. The invention further teaches that the base material is modified by mixing it with additives to improve rigidity of the needle part, to improve the flowability of the mixture and reduce its surface tension to better fill thin parts and fill out sharp corners in the molds during the molding process and to improved the surface characteristic whereby flow of the fluid conveyed by the finished molded-in-one-piece tip and its needle part is facilitated.

XX.

At least one flow facilitator is PTFE or polytetratfluoroethylene employed in submicron sizes in concentrations between 1 and 10 weight percent in a blend with a polyolefin such as polyethylene. It is believed that the PTFE is substantially insoluble in the polyolefin and is distributed within a matrix. Other flow facilitators are modified forms of talc, micron sized glass beads and calcium carbonate. Others are Ticona “Ceramer TM” a Polyphenylene Sulphone (PPSO₂) and DuPont Fluoroguard “TM. Flow facilitators both improve the quality of the finished tip but also reduce flow pressure drop within the tip as it applied.

XXI.

The invention teaches that mineral particles of extremely small particle size, when mixed uniformly into the base thermoplastic sharply increases the rigidity of the needle part. The prior art teaches that, even when polypropylene needle as fabricated separately, using the best techniques, they are still very flexible. Manufacturers turn this fault into a sales tool by describing their glued-in-place polypropylene needles as ‘flexible.’ ( Page 23, EFD Company catalog; 977 Waterman Ave, East Providence, R.I. 02914-1378; See Disclosure Document.)

XXII.

Some minerals that are available in sub-micron particle sizes are generally called nanomers. One preferred nanomer increases the rigidity (flexural modulus) of the needle part by a factor of about 5 when used in a concentration of 5 weight percent in a polypropylene or Nylon 6 or polyethylene composite without negatively affecting other desirable properties of the matrix. That material is an onium modified montmorillonite having a dry particle size of 15 to 50 microns. One source for material of this type is Nanocor; 1500 W. Shure Drive; Arlington Heights, Ill. 60031. Other sources are available under different names. The material has been recommended for use in thermoplastic applications requiring increased fire resistance such as electrical junction boxes, in vapor tight containers as soda bottles. While the nanomer has been found to increase polyolefin rigidity when incorporated therein as a matrix, no suggestion of the use of such material or matrix for tubing of any kind or in a hypodermic tip has been observed in any related material. Other nanomers having similar properties as uses but not applied before in the production of hypodermic tips are forms of talc, micron sized glass beads and forms of calcium carbonate.

XXIII.

Comparing FIGS. 2A and 2B discloses significant physical differences that generate significant performance and cost differences. Passages 40 of small interior dimension exhibit resistance to flow proportional to their length. For a given external needle length 36, the prior art glued-in-place needle part 42 has an overall restrictive length 46 while the integrally molded needle part 22 has an overall restrictive length 36, a length difference 52. In a typical tip construct the restricted passage length difference 42 is of the order of 0.25 inches. In a production run of one million pieces, not an unusual number, this may amount to a total length of 20,800 feet, representing a not inconsiderable cost. Further, the abrupt inlet to the prior art, cemented-in-place needle 42, generates an orifice-like condition that causes high turbulence and therefore high resistance to flow. By contrast the streamlined transition 34 between the integrally molded needle part 22 and the barrel provides low turbulence and very much lower resistance to the flow of fluids resulting in less pressure loss, lower required inlet pressures and superior, more consistent output with better defined droplet shape and reduced slumping, stringing and tailing between the outlet tip 39 of the needle part and the material deposited.

XXIV.

FIG. 3A is a crossectional partial illustration of the composite mold construction employed to produce the tips of FIGS. 1A, 1B and 2B and more generally 4,A,B,C and 5A,B, and C. In FIG. 3A the lower mold portion 54, employed to form the periphery of the needle part, is made of porous steel. Porous steel is a mold material formed of sintered steel particles. It is available from a number of manufacturers both USA based and foreign. Among these are International Mold Steel, Inc.; Florence, Ill. 41042; Thyssen Specialty Steels; Carol Stream, Ill.; Carrs Tool Steel, Ltd; Tipton, West Midlands, DY4 8XQ, United Kingdom; and ToolVac Engineering AB; Perstorp, Sweden. The material is employed as the restrictive element in precision flow restrictors, as filter in chemical and biological research and as molds for both themoplastic and thermoset materials.

XXV.

The porous steels are made with pore sizes ranging from a fraction of a micron to about 40 microns. For molding the needle part of hypodermic tips, there is a compromise between venting effectiveness, surface smoothness and clogging of the pores. The most effective pore size has been found to be between 1 and 5 microns. Molds for the needle part formed of steel having a pore size in this range require minimum cleaning from clogging effects and still provide effective venting.

XXVI.

Referring again to FIG. 3A the cylindrical cavity 64 for the needle part 22 is formed by Electrostatic Discharge Machining or EHD. The mold interior is polished with a reciprocating motion, called draw polishing. The mold interior is not plated. Within the cylindrical cavity is positioned mandril 60 whose outside diameter determines the inside diameter 40 of the needle part. Mandril 60 is positioned precisely along the central axis 125 (FIG. 6D) of the needle part. The end 62 of the mandril 60 is tapered to fit precisely into core 72. Core 72 is formed to determine the inner shape of barrel 24 and is formed with cavity 78 (FIG. 3C) to mate with and precisely position the mandril end 62 within the cavity 64 for the needle part, thereby assuring substantially concentric positioning of the bore 40 of the needle part and substantially uniform wall thickness of the needle part. Mandril 60 is mounted into base plate 58 (FIG. 3B) which plate also acts to close the end of the needle part mold 64. Channel 88 is a vent provided to allow air and mold gases displaced by the entering polymer mixture to leave the mold, thereby facilitating complete filling of the mold.

XXVII.

The upper mold portion 56 is formed of ordinary tool steel. Mold part 56 is employed to form the exterior of barrel 24 and the Luer or other connector at end 30 of the tip. The upper mold part 56 is provided with passages 76 called gates for providing entry of the pressurized molten polymer matrix. One or more upper gates, not shown, provide polymer delivery to the tip body 24. The lower gate 70 positioned in the mold for the needle part as shown in FIG. 3A provides polymer to the needle part. The number of gates required to properly fill the needle part depends on the temperature difference between the mold and the melting point of the polymer, the wall thickness of the needle part, the lubricity of the mold and the fluidity or viscosity of the molten polymer. The number of gates can be reduced when the porosity of the needle part is high but at the expense of mold clogging and surface roughness of the needle part.

XXVIII.

Mandril 60, is coated with a Titanium Nitride coating as is the exterior 140 of barrel mandril 72 and the interior 141 of barrel mold 56 (FIG. 3C). The purpose of the Titanium Nitride coat is to resist wear and to facilitate the removal of the cast tip after solidification of the polymer. Other coatings are available for this purpose such as Chromium Nitride, Chromium Carbide, Titanium Carbide and Tungsten Carbide, though for small parts, Titanium Nitride is most satisfactory. Other hard surface coatings include but are not limited to lamellar Tungsten disulfide, PTFE impregnated Nickel Phosphorous, Chromium metal, Teflon and Boron carbide

XXIX.

Referring now to FIGS. 3A and 3C, the junction line 78 between the porous steel mold 54 for the needle part and the steel mold 56 for the barrel does not separate during the casting process. This mold is made with a parting line at the Luer 28. This parting line is not shown.

XXX.

FIG. 3D is an enlarged view of the juncture between the mandril 60 for the bore of the needle part and the base plate 58. The shape of the enlargement is selected to define the design of the output of adhesive, flux or other material from the end 39 of the needle part 22 during the course of its use. The enlarged part 66 of the mandril 60 can be formed to provide required shapes such as a square, star, a cross (FIG. 3E) or any similar shape within the interior end of the needle part. The viscous material flowing from the end 39 of the needle part assumes the preformed shape of mandril end 66 leaving a deposit of the desired shape.

XXXI.

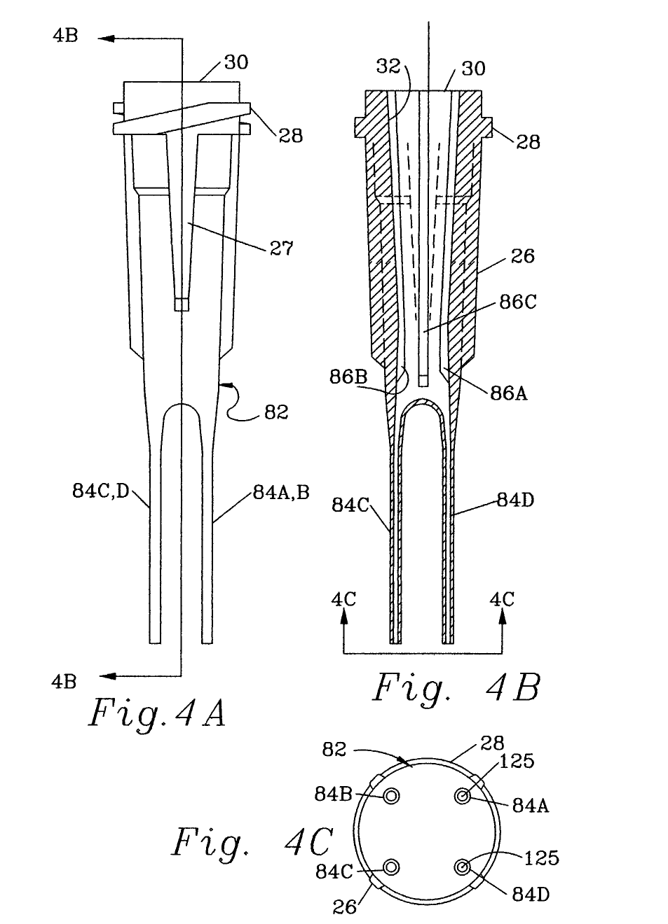

Referring now to FIGS. 4A, 4B and 4C, there is shown in FIG. 4A a side elevation of a hypodermic tip 82 whose upper barrel construction is substantially the same as that of tip 20 (FIG. 1A). However, instead of providing a single needle part 22, there are provided four needle parts 84A, 84B, 84C and 84D each having central axis' 125 (FIGS. 4C, 6D) that are parallel to each other and are placed at the corners of a four-sided polygon, in this case a square, though other polygon shapes and other numbers of parallel needle parts are taught.

XXXII.

Positioned within the barrel 32 are turbulator devices 86. While three turbulators 86A, 86B and 86C are identified in FIG. 4B, other numbers and sizes of turbulators may be employed. The turbulators 86 are intended to create turbulent condition within the barrel so that flow and particle distribution to the multiple needle parts is equalized, thereby allowing the end 39 (FIG. 1A) of each needle part to deliver substantially the same quantity of fluid as its neighbor.

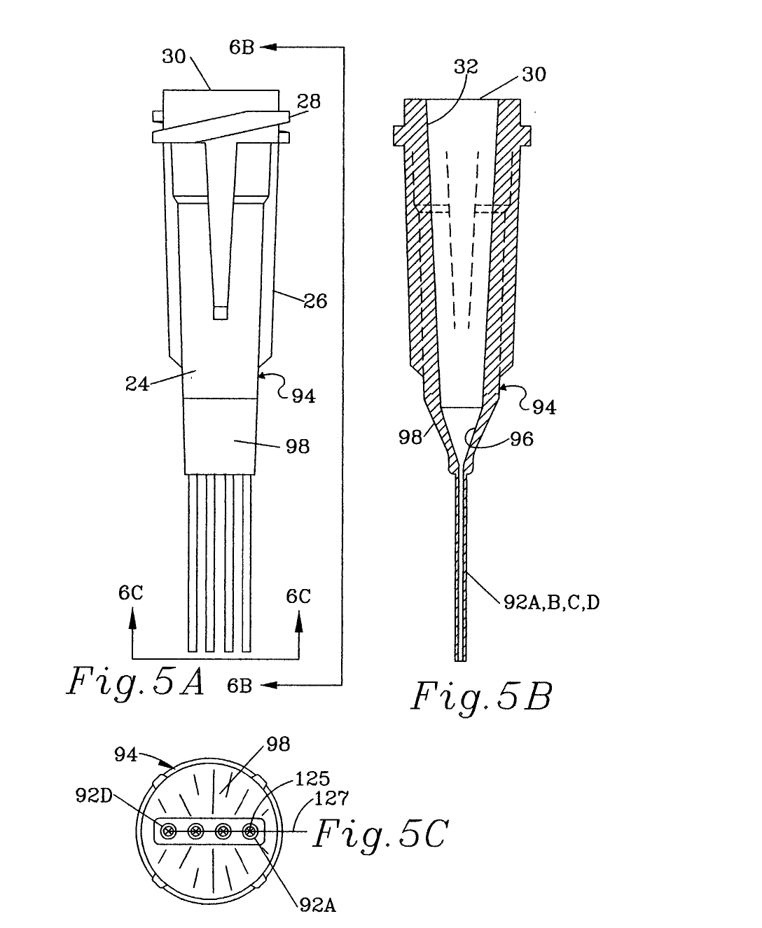

In FIGS. 5A, 5B and 5C the object is to display a tip 94 having multiple needle parts 92A, 92B, 92C and 92D, each positioned with its centerline substantially parallel to the centerline of the others, but with all centerlines arranged along a straight line 127. The barrel part 24 of tip 94 is numbered the same as and is intended to indicate that the barrel part of tip 94 has substantially the same shape as the barrel part of tip 20 in FIG. 1A.

XXXIII.

The tip 94 includes a transition 98 having a streamlined interior 96 to provide substantially uniform flow to the needle parts 92.

XXXIV.

Referring now to FIG. 6A there is shown a crossectional view of a mold 100 where the parting line 122 lies along the centerline of the bores of the barrel part and the needle part. In 6A the porous steel mold portion 102 and 104 for forming the needle part of the tip are separate and have a parting line along centerline 122. In like manner the mold portions 111 and 110 forming the upper and lower portions of the mold for the body portion are formed of substantially the same material as the mold portion 56 for the body portion in FIG. 3A. However, the mold for the body portion is in two parts 110 and 111 and is split along the parting line 122 that is also the centerline of the tip.

XXXV.

Referring to FIG. 6B there is shown an enlarged central part of the mold of 6A. This enlarged view shows clearly that the mandril portion for the body part 112 and the mandril portion 113 for the needle part have been formed as one piece.

XXXVI.

Referring now to FIG. 6C there is shown a crossection of a mold having mandril 113, upper part 102 and lower part 104 for a needle part 124 (FIG. 6D). The mold produces a needle part 124 (FIG. 6D) having internal groves C and interior ribs D corresponding to the ribs 130 and the grooves 132 formed in the mandril 113 for the needle part and exterior grooves a and ribs B formed by the corresponding ribs 134 and grooves 136 formed in the mold.

XXXVII.

While the ribs D and B in the interior and exterior of needle part 124 are the preferred construction, special requirements may demand both ribs on the interior or both grooves or one and the other or one and none, for instance providing a smooth needle part exterior and a ribbed interior. The outer portions 102 and 104 for the mold of the needle part are split at the parting line 112 that lies along the centerline 125 of the needle part.

XXXVIII.

In FIG. 6E there is shown the crossection of a needle part 138 that has been formed with flat longitudinal faces 126 on the exterior whereby the crossection is an octagonal polygon. The interior has been formed with flat longitudinal panels 128 whereby the crossection is a hexagonal polygon. The designs providing ribs and or polygonal crossections can provide stiffens of the needle part beyond those that can be provided by the polymer material alone. The external and internal dimensions of the polygonal crossections shall be identified as the diameters and shall be the average dimensions between opposing flat panels. The inside diameter of the needle part may be identified as ‘the bore.’

XXXIX.

A preferred process for forming a one piece hypodermic tip the tip including a barrel having a first and a second end, a connector part, the connector part being molded integral with the barrel first end and at least one needle part the needle part being molded integral with the barrel second end, said needle part having a uniform diameter and a substantially uniform bore communicating with the barrel, a length equal to or greater than 0.4 inches and a diameter equal to or less than 0.072 inches includes at least some of the following steps:

a. Providing a matrix of thermoplastic resin having distributed therethrough at least 5% clay particles such as Nanoclay, described above;

b. Adding a flowability improver such as Polyphenylene Sulphone (PPSO₂)to the matrix;

c. providing a mold having a first part for forming the barrel and connector end and a second part for forming the needle part, said second part being formed of a porous metal such as porous steel, described above;

d. Reducing the pressure in the mold by evacuation to a pressure not higher than 50% of then atmospheric pressure;

e. heating the matrix above the melting point of the thermoplastic component;

f. Heating the mold to a temperature less than the melting point of the matrix;

g. And injecting the matrix under pressure into said heated evacuated mold.

XL.

### SUMMARY AND PREAMBLE TO THE CLAIMS

From the foregoing description, it can be seen that the present invention comprises a new and unique construction for a hypodermic tip, a unique mold construction therefore and a unique polymer matrix composition for forming such a hypodermic tip whereby the needle part of the tip has rigidity and strength not achieved by the prior art. It will be appreciated by those skilled in the art that changes could be made to the embodiments described in the foregoing description without departing from the broad inventive concepts embodies therein. It is understood, therefore, that this invention is not limited to the particular embodiment or embodiments disclosed, but is intended to cover all modifications which are within the scope and spirit of the invention as defined by the appended claims, its elements, and equivalents thereof as described in the above specification.

I claim:

1. A one piece hypodermic tip, said tip comprising a barrel having a first and a second end, a connector part, the connector part being molded integral with the barrel first end and a needle part, said needle part being molded integral with the barrel second end, said needle part having a length, a substantially uniform outside diameter along the length and a substantially uniform bore communicating with the barrel, further providing that the length is equal to or greater than 0.4 inches and the diameter equal to or less than 0.072 inches.

2. A one piece molded hypodermic tip as recited in claim 1 further providing that the needle part has an external diameter between 0.030 and 0.034 inches and a bore equal to or greater than 0.006 inches.

3. A one piece hypodermic tip as recited in claim 2 where the tip is formed of a thermoplastic material.

4. A one piece hypodermic tip as recited in claim 2, further providing that the tip is formed of a matrix comprising a thermoplastic material having a melting point and substantially uniformly distributed solid particles having a higher melting point than the melting point of the thermoplastic material.

5. A one piece hypodermic tip as recited in claim 4 where the solid particles are derived from materials selected from the group consisting of mineral clay, PTFE, talc, micron sized glass beads and calcium carbonate.

6. A one piece hypodermic tip as recited in claim 4, further providing that the solid particles are a form of mineral clay.

7. A hypodermic tip as recited in claim 4, further providing that the tip includes two or more needle parts each having a central axis.

8. A hypodermic tip as recited in claim 7, further providing that the central axis of all tips are positioned substantially parallel to each other.

9. A hypodermic tip as recited in claim 8, further providing that the tip includes at least three needle parts and that their axis' are positioned along a straight line.

10. A hypodermic tip as recited in claim 8, further providing that the tip includes three or more needle parts and that their axis' are positioned at the interstices of a polygon.

11. A hypodermic tip as recited in claim 4, further providing that the matrix includes a flow enhancing material.

12. A hypodermic tip as recited in claim 11 where the flow enhancing material is selected from the group consisting of Polyphenelene Sulphone (Ticona “Ceremer) and Dupont “Fluoroguard.”

13. A hypodermic tip as recited in claim 4 further providing that a crossection of the needle part has an exterior polygonal outline.

14. A hypodermic tip as defined in claim 4 further providing that a crossection of the needle part has an interior polygonal outline.

15. A hypodermic tip as defined in claim 4 further providing that the exterior of the needle part includes longitudinal ribs.

16. A hypodermic tip as recited in claim 4 further providing that the interior of the needle part includes longitudinal ribs.

17. A mold for producing a one piece hypodermic tip, the tip comprising a barrel having a first and a second end, a connector part, the connector part being molded integral with the barrel first end and a needle part, said needle part being molded integral with the barrel second end, said needle part having a uniform diameter and a substantially uniform bore communicating with the barrel, a length equal to or greater than 0.4 inches and a diameter equal to or less than 0.072 inches, the mold comprising a first part formed of a first material for molding the barrel and connector part and a second part formed of a second material for molding the needle part.

18. A mold as recited in claim 13 further providing that the second material is porous steel.

19. A mold as recited in claim 14 where the mold first part has an interior surface and further providing that said interior surface has positioned thereon a flow facilitating coating.

20. A mold as recited in claim 14 further providing that the tip has a longitudinal axis substantially coinciding with the axis of a needle part and an extension thereof, a parting plane along which the parts of the mold are separated for removal of the molded tip, and further providing that said parting plane is positioned substantially adjacent to the connector end of the barrel and further positioned so that the tip axis is substantially perpendicular to said parting plane.

21. A mold as recited in claim 14 further providing that the tip has a longitudinal axis substantially coinciding with the axis of a needle part and further providing a parting plane along which the parts of the mold are separated for removal of the molded tip and that the parting plane traverses the entire length of said axis.

22. A mold as recited in claim 14 further providing at least two needle parts.

23. A mold as recited in claim 18 further providing at least three needle parts each positioned at an interstice of a polygon.

24. A mold as recited in claim 19 further providing that the needle part is formed to produce a needle part having a polygonal crossection.

25. A process for forming a one piece hypodermic tip the tip comprising a barrel having a first and a second end, a connector part, the connector part being molded integral with the barrel first end and at least one needle part, said needle part being molded integral with the barrel second end, said needle part having a uniform diameter and a substantially uniform bore communicating with the barrel, a length equal to or greater than 0.4 inches and a diameter equal to or less than 0.072 inches, the process comprising the steps of:
providing a matrix of thermoplastic resin having distributed therethrough at least 5% clay particles;
providing a mold having a first part for forming the barrel and connector end and a second part for forming the needle part, said second part being formed of a porous metal;
heating the matrix above the melting point of the thermoplastic component and injecting under pressure the matrix into said mold.

26. A process as recited in claim 20 further providing the step of heating the mold to a temperature lower than the melting point of the matrix.

27. A process as recited in claim 21, further providing the step of lowering the ambient pressure within the mold to approximately 50% of the then absolute atmospheric pressure before injecting the matrix into the mold.
